(19) United States
(12) Patent Application Publication
(10) Pub. No.: US 2009/0093028 A1
(43) Pub. Date: Apr. 9, 2009

(54) APPARATUS AND METHODS FOR TREATING BIOMASS

(76) Inventors: JOY DORAN PETERSON, Athens, GA (US); MARK EITEMAN, Athens, GA (US); SARAH KATHERINE BRANDON, Raleigh, NC (US)

Correspondence Address: MUETING, RAASCH & GEBHARDT, P.A., P.O. BOX 581336, MINNEAPOLIS, MN, 55458-1336, US

(21) Appl. No.: 12/245,146

(22) Filed: Oct. 3, 2008

Related U.S. Application Data
(60) Provisional application No. 60/997,761, filed on Oct. 5, 2007.

(51) Int. Cl. C12P 19/02; C12M 1/00; C12P 1/00

(52) U.S. Cl. 435/105; 435/287.5; 435/277

(57) ABSTRACT

An apparatus for treating plant biomass is described herein. Generally, the apparatus includes a pressurizable vessel and a heating element in thermal communication with the vessel. Also described herein are methods of treating plant biomass. Generally, the methods include heating plant biomass under conditions effective to at least partially depolymerize hemicellulose and/or cellulose present in the plant biomass. In one embodiment, the method includes heating a plant biomass substrate to a temperature of 230° C. for two minutes at a pressure of 57 psig.

### CROSS-REFERENCE TO RELATED APPLICATION

This application claims priority to U.S. Provisional Patent Application Ser. No. 60/997,761, filed Oct. 5, 2007.

### BACKGROUND

Renewed interest in alternatives to petroleum products, especially for liquid transportation fuels, has increased demand for ethanol. Producing fuel from renewable resources such as grasses is desirable because of the large quantities of biomass available. Established forage grass crops such as, for example, switchgrass, bermudagrass, and napiergrass initially were bred for increased biomass production as animal feedstocks, but the increased biomass of these (and other) grasses makes them attractive sources for ethanol production as well. Bermudagrass (Cynodon dactylon) is grown on 10-15 million acres in the southern United States. Tifton 85 (T85) is a hybrid between Tifton 68 and PI 290884 from South Africa. This grass is hardy and produces significantly more dry matter than other bermudagrass cultivars.

Efficient conversion of a plant material substrate to ethanol typically involves pretreating a substrate prior to enzymatic hydrolysis, making the substrate more available for enzymatic action. Once complex carbohydrates such as, for example, hemicellulose and cellulose in the substrate are converted to monomeric sugars by enzymatic hydrolysis, these sugars can be more easily fermented by microorganisms to produce ethanol.

In addition, grasses can include low molecular weight aromatic constituents linked to otherwise fermentable sugars—e.g., phenolic acids ester-linked to arabinose. Such compounds can occur in non-lignified parts of plant cell walls. Treatments designed to separate the fermentable sugars from the aromatic constituents could enhance fermentation yields and provide a valuable co-product.

Thus, methods in which complex carbohydrates of plant biomass are converted to fermentable sugars are desirable.

### SUMMARY OF THE INVENTION

Pretreatments involving extraction of plant biomass can provide an effective way to pretreat cellulosic material by beginning disruption of cellulose and/or hemicellulose prior to enzymatic hydrolysis. This treatment generally involves exposing a plant biomass substrate to highly pressurized water at high temperatures.

Accordingly, in one aspect, the present invention provides an apparatus useful for performing such treatments. Generally, the apparatus includes a vessel that includes a chamber into which a biomass sample may be placed and a cover that may be capable of forming a liquid-tight and pressure-tight seal. The vessel further includes an inlet port providing fluid communication between the chamber and a pressure source, a reversibly openable pressure outlet in fluid communication with the chamber, a temperature detector in thermal communication with the chamber and in communication with a display, and a pressure detector in communication with the chamber and in communication with a display. The apparatus further includes a heating element in thermal communication with the external surface of the vessel wall.

In some embodiments, the apparatus may be automated by connecting one or more functional components of the apparatus to a process controller such as, for example, a computer. In other embodiments, one or more functional components may be configured for manual operation.

In some embodiments, the apparatus may further include one or more of the following: a timer, a pH detector, a fluid outlet in fluid communication with the chamber, and a condenser that is, if present, in fluid communication with the fluid outlet.

In another aspect, the present invention provides a method of processing plant biomass. Generally, the method includes providing plant biomass comprising at least one of: cellulose and hemicellulose; and heating the plant biomass under conditions and for a time sufficient to at least partially depolymerize one or more of: the hemicellulose and the cellulose, thereby yielding processed plant biomass; wherein the time is at least one minute; and wherein the conditions comprise: a volume of liquid medium so that the plant biomass is provided in an amount of at least about 0.01% w/v solids up to about 50% w/v solids; a temperature of at least about 180° C.; and pressure of at least about 200 psia.

In another aspect, the invention provides a method of pretreating plant biomass. Generally, the method includes providing plant biomass comprising at least one of: cellulose and hemicellulose; pretreating the plant biomass under conditions and for a time sufficient to at least partially depolymerize one or more of: the hemicellulose and the cellulose, thereby yielding pretreated plant biomass, wherein the time is at least one minute, and wherein the conditions include: a volume of liquid medium so that the plant biomass is provided in an amount of at least about 0.01% w/v solids up to about 50% w/v solids, a temperature of at least about 180° C., and pressure of at least about 200 psia; and subjecting the pretreated plant biomass to additional treatment.

In yet another aspect, the present invention provides a method of preparing a plant biomass substrate for fermentation. Generally, the method includes providing a plant biomass substrate; heating the plant biomass substrate to a predetermined temperature of at least about 180° C. to no greater than about 300° C.; pressurizing the plant biomass substrate to a predetermined pressure of at least about 200 psia to no greater than about 1000 psia; maintaining the predetermined temperature and the predetermined pressure for a period of at least one minute and no greater than about 120 minutes; cooling and depressurizing the plant biomass substrate; providing an enzymatic composition comprising at least one enzyme capable of converting at least one of hemicellulose and cellulose to monomeric sugar; and incubating the cooled plant biomass substrate with the enzymatic composition under conditions effective for the enzyme composition to convert at least one of hemicellulose and cellulose to monomeric sugars.

The above summary of the present invention is not intended to describe each disclosed embodiment or every implementation of the present invention. The description that follows more particularly exemplifies illustrative embodiments. However, embodiments other than those expressly described are possible and may be made, used, and/or practiced under circumstances and/or conditions that are the same or different from the circumstances and/or conditions described in connection with the illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 is a schematic diagram of a pressurized hot water hydrolysis system.

FIG. 2 illustrates temperature and pressure profiles for a pressurized hot water hydrolysis treatment performed for two minutes at a set point temperature of 200° C.

FIG. 3 shows enzyme digestibility of bermudagrass following pressurized hot water hydrolysis treatment for two minutes as a function of reaction temperature. The curve depicts the model prediction of sugar yields as a function of temperature. The bar represents the sugar yield of a sample of untreated bermudagrass.

FIG. 4 shows the average reducing sugar concentration and ethanol production over the course of fermentations of T85 Tifton bermudagrass samples that were untreated (⋄ and ♦), PBHW-treated at 200° C. (Δ and ▴), and PBHW-treated at 230° C. (□ and ▪). Fermentations were performed at 35° C. in an immersion circulator for five days at 10% solids. The −24 hr. time point corresponds with the beginning of the 24 hour enzyme hydrolysis performed at 45° C. The actual fermentation began with bacterial inoculation at time 0 hr. Reducing sugars are removed at the same rate that ethanol is produced.

### DETAILED DESCRIPTION OF ILLUSTRATIVE EMBODIMENTS

The present invention relates to methods for processing plant biomass including, for example, pretreating plant biomass so that the plant biomass is more susceptible to subsequent downstream treatments and/or processes such as, for example, enzymatic hydrolysis. Generally, the methods include pressurized batch hot water (PBHW) treatment—e.g., heating in the presence of water in a pressurized environment.

The present invention further provides an apparatus capable of performing the PBHW treatment. The apparatus includes a vessel that includes a pressurizable chamber and a mechanism by which contents of the chamber—e.g., plant biomass and water—may be heated at a prescribed pressure, to a prescribed temperature, for a prescribed length of time.

PBHW pretreatment is promising for increasing the digestibility of complex carbohydrates in plant biomass for subsequent processing and/or treatments. The non-flow-through PBHW reactor described herein is reliable and effective; pressure and temperature were held constant over the reaction time and significant dissolution of complex carbohydrates occurred as measured by enzymatic hydrolysis.

### DEFINITIONS

“Agricultural residue” refers to plant material remaining after harvesting a crop, including, for example, leaves, stalks, and/or roots.

“Forest residue” refers to material not harvested from commercial hardwood and/or softwood logging sites, and/or material resulting from forest management operations including, for example, precommercial thinning and removal of dead or dying trees.

“High cellulose” refers to plant material that comprises at least 50% cellulose such as, for example, cotton and wood pulp.

“High pectin” refers to plant material that comprises at least 0.5% pectin such as, for example, apples, apricots, citrus, sugarbeets, and other fruits and vegetables.

“Percent solids (% w/v solids)” refers to the w/v ratio of plant biomass solids to a liquid medium that includes, for example, water, or liquid growth media such as, for example, Luria-Bertani (LB) media or Tryptic soy broth.

“Psia” refers to pound-force per square inch absolute, a unit of pressure relative to a vacuum. At sea level, the Earth's atmosphere exerts a pressure of approximately 14.7 psi. Thus, at sea level, psia=psig+14.7 psi.

“Psig” refers to pound-force per square inch gauge, a unit of pressure relative to the surrounding atmosphere. At sea level, the Earth's atmosphere exerts a pressure of approximately 14.7 psi. Thus, at sea level, psig=psia−14.7 psi.

The term “and/or” means one or all of the listed elements or a combination of any two or more of the listed elements.

The terms “comprises” and variations thereof do not have a limiting meaning where these terms appear in the description and claims.

Unless otherwise specified, “a,” “an,” “the,” “one or more,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

In one aspect, the present invention provides an apparatus suitable for pretreating plant biomass as described herein. Generally, the apparatus includes a vessel that includes a chamber into which plant biomass substrate may be placed. The chamber may be closed and the vessel pressurized. The apparatus further includes a heating element capable of heating the contents of the vessel. Various functions of the apparatus may be performed manually. Alternatively, in certain embodiments, the apparatus may include a process controller and/or data acquisition unit (e.g., a computer) configured to automate one or more functions of the apparatus.

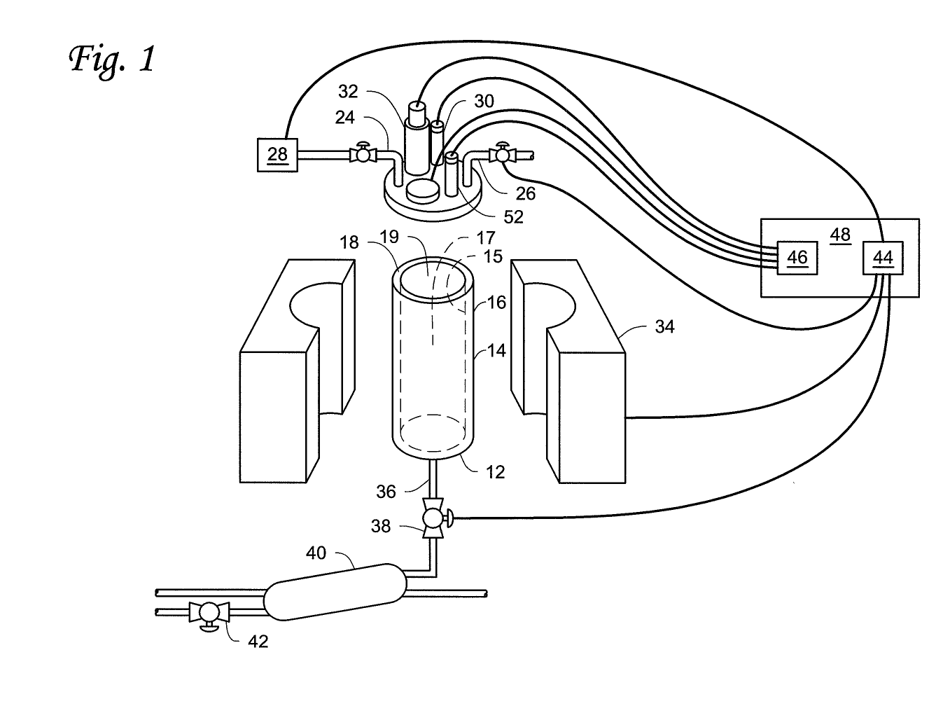

Referring to FIG. 1, the apparatus 10 includes a vessel 12 into which plant biomass may be placed for treatment. The vessel 12 includes at least one wall 14. The at least one wall 14 includes an internal surface 15 and an external surface 16. The internal surface 15 defines a chamber 17. The external surface 16 defines an edge 18 that, in turn defines an opening 19 to the chamber 17.

A cover 20 is configured to sealably close the chamber opening 17. The cover 20 may include a deformable material such as, for example, a polymeric (e.g., rubber or plastic) material capable of forming a seal with the edge 18 of the vessel 12. Alternatively, the vessel 17 can include the deformable material capable of forming a seal with the cover 20. The cover 20 may be secured to the vessel 17 during operation by any suitable method. Suitable methods include, for example, the use of clips, clamps, complementary threads on the cover and vessel, etc.

In some embodiments, the apparatus 10 can include a receptacle that may be used to contain a sample of plant biomass within the chamber 17 during operation. The receptacle may be any suitable form. In some embodiments, the receptacle may be liquid permeable such as, for example, mesh, a net, a basket, or the like.

The apparatus 10 includes an inlet port 24 providing fluid communication between the chamber 17 and a pressure source 28. The apparatus 10 also includes an outlet 26 in fluid communication with the chamber 17. The outlet 26 may be reversibly closable so that in the closed position it is capable of limiting release of pressure from the chamber 17 during operation, while in the open position it is capable of permitting the release of pressure from the chamber 17. The apparatus also includes a temperature detector 30 in thermal communication with the chamber 17. When the apparatus is in operation, the temperature detector 30 may be used to monitor the temperature inside the chamber 17. The temperature detector 30 is in communication with a display, which may form a portion of the temperature detector 30. Alternatively, the display may be remote from the temperature detector 30. The apparatus also includes a pressure detector 32 in communication with the chamber 17. When the apparatus is in operation, the pressure detector 32 may be used to monitor the pressure inside the chamber 17. The pressure detector 32 is in communication with a display, which may form a portion of the pressure detector 32. Alternatively, the display may be remote from the pressure detector 32. The apparatus also may include a pH detector 52 in communication with the chamber 17. When the apparatus is in operation, the pH detector 52 may be used to monitor the pH inside the chamber 17. The pH detector 52 may be in communication with a display, which may form a portion of the pH detector 52. Alternatively, the display may be remote from the pH detector 52. Each of the inlet port 24, outlet 26, temperature detector 30, pressure detector 32, and pH detector 52 may, independent of each of the others, be incorporated into the cover 20 or, alternatively, incorporated into a portion of the at least one vessel wall 14.

The apparatus 10 also includes a heating element 34 in thermal communication with the external surface 16 of the at least one vessel wall 14. The heating element 34 may be of any type adequate to heat the contents of the chamber 17 sufficiently to practice the methods described herein.

In some embodiments, the apparatus 10 further includes a fluid outlet 36 in fluid communication with the chamber 17. After pretreating the plant biomass according to a method described herein, fluid may be removed from the chamber 17 via the fluid outlet 36. The fluid outlet 36 may be integrated into the vessel 17 or, alternatively, integrated into the cover 20. The fluid outlet 36 may include a valve 38 so that the fluid outlet 36 may be reversibly closable. Thus, the fluid outlet 36 may be reversibly closable so that in the closed position it is capable of limiting release of fluid from the chamber 17 during operation, while in the open position it is capable of permitting the removal of liquid from the chamber 17.

In some embodiments, the fluid outlet 36 can provide fluid communication between the chamber 17 and a condenser 40. The condenser 40 can include a condenser fluid outlet 42 from which condensate may be collected during operation.

The apparatus 10 can further include a process controller 44 in communication with one or more of the heating element 34, pressure source 28, pressure outlet 26, and fluid outlet 36. In operation, the process controller 44 may control one or more functions of the apparatus 10. For example, the process controller 44 may control the heating element 34 and, therefore, heating, cooling, and maximum temperature of the chamber 17 and, therefore, its contents. As another example, the process controller 44 may control pressure inside the chamber 17 by controlling the pressure source 28 and/or the pressure outlet 26. As yet another example, the process controller 44 may control fluid movement out of the chamber 17 by controlling the fluid outlet 36.

In some embodiments, the apparatus 10 can further include a data acquisition unit 46 in communication with the temperature detector 30, pressure detector 32, and/or pH detector. In some embodiments, the data acquisition unit 46 can include one or more of the displays capable of displaying data from the temperature detector 30, the pressure detector 32, and pH detector, respectively. Thus, in some embodiments, the displays may be distinct components of the apparatus 10. However, in other embodiments, the displays may be combined in, or indeed be, the same component of the apparatus 10.

In some embodiments, the apparatus 10 can include a timer 50, which may be in communication with the data acquisition unit.

In some embodiments, the process controller 44 and/or the data acquisition unit 46 may include a computer 48. In such embodiments, a single computer 48 can be the process controller 44 and/or the data acquisition unit.

In another aspect, the present invention provides methods of processing plant biomass. In some circumstances, the processing method may be considered a pretreatment method in which the plant biomass is treated as described herein prior to being subjected to subsequent additional treatment. For example, a method as described herein may be considered to be a pretreatment method in which plant biomass is prepared for fermentation by one or more microbes. Unless otherwise specified, descriptions of aspects of the methods that follow apply to any embodiments of the methods, whether considered a processing method, pretreatment method, or preparation method.

Generally, the methods include heating plant biomass in a pressurized environment such as, for example, a pressurized environment provided in the chamber 17 of the apparatus 10 described above. The plant biomass may be any suitable plant biomass for which the treatment described herein may be desired. Suitable plant biomass substrates include, for example, agricultural residue, forest residue, waste stream residue, and/or a mixture or combination thereof. Agricultural residue can include, for example, plant material remaining after harvesting a crop including, for example, leaves, stalks, roots, and/or mixtures or combinations thereof. Exemplary agricultural residues includes, for example, leaves, grass, corn stover, corn cob, sugar cane stalk, sugar cane bagasse, sorghum stalk, sorghum bagasse, and/or mixtures or combinations thereof. Forest residue can include, for example, material not harvested from commercial hardwood and/or softwood logging sites, and/or material resulting from forest management operations including, for example, precommercial thinning and removal of dead or dying trees. Waste stream plant biomass can include, for example, recycled paper.

In other embodiments, the plant biomass may be, for example, a grassy substrate, a high cellulose substrate, a woody plant substrate, a high pectin substrate, and/or mixtures or combinations thereof. As used herein, grassy substrates refer to plant biomass substrates that are grasses and can include, for example, timothy grass, bermudagrass, napier grass, sorghum, and/or switchgrass, and mixtures and/or combinations thereof. Grassy substrates can include, for example, one or members of Sorghum spp. (e.g., Sorghum bicolor), Miscanthus spp. (e.g., Miscanthus sinesis or), Saccharum spp. (e.g., Saccharum ravennae), Zea spp. (e.g., Zea mays), Panicum spp. (e.g., Panicum virgatum), Cynodon spp. (e.g., Cynodon dactylon, Cynodon transvaalensis, Cynodon magennissii, etc.), Phleum spp. (e.g., Phleum pratense), or Pennisetum spp (e.g., Pennisetum purpureum). Grassy substrates also can include hybrid grasses such as, for example, Miscanthus giganteus (Miscanthus×giganteus) or hybrids of Miscanthus spp. and Saccharum spp. (e.g., miscane and/or energycane). High cellulose substrates can include plant material that includes at least 50% cellulose such as, for example, cotton (Gossypium spp.) and wood pulp from, for example, pine (Pinus spp.) or poplar (Populus spp.). High pectin plant biomass substrates can include plant material that includes at least 0.5% pectin such as, for example, apples, apricots, citrus, sugarbeets, and other fruits and vegetables.

In some cases, the plant biomass may be reduced in size so that, for example, it can fit into a portion of an apparatus (e.g., a vessel) in which it will be subjected to a method as described herein. The biomass size may be reduced by, for example, slicing, cutting, chopping, chipping, mulching, or otherwise reducing the size of individual pieces of the plant biomass.

The method includes heating the plant biomass substrate in the presence of a liquid medium that includes, for example, water (e.g., distilled water, very dilute acid, very dilute base, etc.). The conditions under which the plant biomass substrate is treated may be sufficient to at least partially depolymerize hemicellulose and/or cellulose in the plant biomass substrate. In some cases, the conditions may be sufficient to depolymerize, for example, at least about 20% of the hemicellulose in the plant biomass substrate such as, for example, at least about 25%, at least about 30%, at least about 40%, at least about 50%, at least about 60%, or at least about 75% of the hemicellulose in the substrate. In some cases, the conditions may be sufficient to depolymerize at least about 5% of the cellulose in the plant biomass substrate such as, for example, at least about 8%, at least about 10%, at least about 11%, at least about 12%, at least about 15%, or at least about 20% of the cellulose in the substrate. In some embodiments, the depolymerization of hemicellulose and/or cellulose can result in the release of fermentable sugars.

The substrate may be heated to a temperature of at least about 180° C. such as, for example, at least about 200° C., at least about 215° C., or at least about 230° C. In some embodiments, the substrate may be heated to a temperature of no more than about 300° C. such as, for example no more than about 250° C., no more than about 230° C., no more than about 215° C., or no more than about 200° C. In certain embodiments, the substrate is heated to a temperature of at least about 200° C. and no more than about 250° C. such as for example, a temperature of at least about 215° C. and no more than 230° C. In one particular embodiment, the substrate is heated to a temperature of about 230° C.

The plant biomass may be provided in a weight to volume ratio with respect to the volume of liquid medium. A suitable substrate to liquid medium ratio may, for example at least about 0.01% w/v solids such as, for example, at least about 1% w/v solids. In some embodiments, a suitable substrate to liquid medium ratio may be for example, up to about 50% w/v solids such as, for example, up to about 15% w/v solids such as, for example, up to about 5% w/v solids. Thus, in some embodiments, a suitable substrate to liquid medium ratio may be, for example, from about 0.01% w/v solids up to about 50% w/v solids such as, for example, from about 1% w/v solids up to about 15% w/v solids. In certain embodiments, the substrate to liquid medium ratio may be, for example, from about 1% w/v solids up to about 5% w/v/ solids. In particular embodiments, the plant biomass substrate may be provided at about 1% w/v solids.

The plant biomass is heated in a pressurized environment—i.e., an environment experiencing a pressure of at least about 200 psia such as, for example, at least about 300 psia, at least about 500 psia, or at least about 700 psia. In some embodiments, the plant biomass is heated in an environment experiencing a pressure of no more than about 1000 psia such as, for example, no more than about 700 psia, no more than about 500 psia, no more than about 400 psia, or no more than about 300 psia.

The plant biomass is heated for a reaction time that is at least about 30 seconds such as, for example, at least about one minute, at least about two minutes, at least about five minutes, or at least about eight minutes. In some embodiments, the plant biomass substrate is heated for a reaction time that is no more than about 20 minutes such as, for example, no more than about 10 minutes, no more than about eight minutes, no more than about five minutes, no more than about two minutes, or no more than about one minute. As used herein, “reaction time” refers to the length of time during which the temperature of the substrate inside the chamber 17 is maintained relatively constant (e.g., ±2° C.) with respect to the set temperature. Reaction time excludes the time during which the temperature of the chamber 17 is heated from ambient or some other non-set temperature to the set temperature. Reaction time also excludes the time during which the chamber 17 and, therefore, its contents (including the plant biomass substrate) are allowed to cool. In some embodiments, the reaction time may be from about one minute to about eight minutes such as, for example, from about 2 minutes to about five minutes. In one particular embodiment, the reaction time may be about two minutes.

In some embodiments, the method may be performed under conditions effective to limit the production of at least one fermentation inhibitor. Typical fermentation inhibitors whose production may be limited in such embodiments include, for example, ferulic acid, para-coumaric acid, furfural, or 5-hydroxymethyl furfural. In some cases, the production of fermentation inhibitors may be limited by maintaining the liquid medium at a pH of at least about 3.0 such as, for example, at least about pH 4.0, such as, for example, at least about pH 4.2. In some cases, the pH of the liquid medium may be maintained at no more than pH 6.0 such as, for example, no more than pH 5.0 such as, for example, no more than pH 4.8. In certain embodiments, the pH of the liquid medium is maintained from about pH 4.2 to about pH 4.8. In particular embodiments, the pH is maintained in the absence of adding a base to the liquid medium.

In certain embodiments, the methods described herein can be considered to be pretreatment methods in which plant biomass substrate is treated prior to subsequent further treatment. In such embodiments, the PBHW-treated (e.g., processed) plant biomass substrate may be cooled and subjected to one or more additional treatments or processes. In other embodiments, the hydrolysate resulting from the PBHW treatment may be subjected to one or more additional processes or treatments. Such subsequent processes include, for example, enzymatic hydrolysis and/or fermentation. Such processes may be performed according to methods known and routine to those skilled in the art. Beall, D. S. et al., Biotechnol. Lett., 14:857-862, 1992; Asghari, A. et al., J. Indust. Microbiol., 21-26, 21-26, 1996; Doran, J. B. et al., Appl. Biochem. Biotechnol., 84-86, 141-152, 2000; Lau, M. W. et al., Biotechnol. Bioeng., 99:529-539, 2008; Leite, A. R. et al., Braz. J. Microbiol., 31:212-215, 2000; Moniruzzaman, M. et al., Biotechnol. Lett., 18, 985-990, 1996; Moniruzzaman, M. et al., Biotechnol. Lett., 20:943-947, 1998; and U.S. Provisional Patent Application Ser. No. 61/097,975, filed Sep. 18, 2008.

Thus, in some embodiments, the method further includes cooling and depressurizing the PBHW-treated plant biomass. The cooled and depressurized plant biomass may then be enzymatically hydrolyzed. The enzymatic hydrolysis can include incubating the PBHW-treated plant biomass with an enzymatic composition under conditions effective for the enzyme composition to convert hemicellulose and/or cellulose in the PBHW-treated plant biomass to monomeric sugars. In many embodiments, the enzymatic composition may be a commercially available enzyme composition. In other cases, the enzyme may be an enzyme composition from Hypocrea jecorina prepared as described in Example 6. In some embodiments, the conditions effective for the enzyme composition to convert hemicellulose and/or cellulose in the PBHW-treated plant biomass to monomeric sugars can include incubating the PBHW-treated plant biomass and the enzyme composition at a temperature of from about 27° C. to about 40° C. such as, for example, from about 27° C. to about 37° C. In certain embodiments, the PBHW-treated plant biomass substrate and the enzymatic composition may be incubated from about 12 hours to about 120 hours.

In some embodiments, practicing the method can result in the production of a hydrolysate. In certain embodiments, the hydrolysate may be collected and subjected to enzymatic hydrolysis as described immediately above.

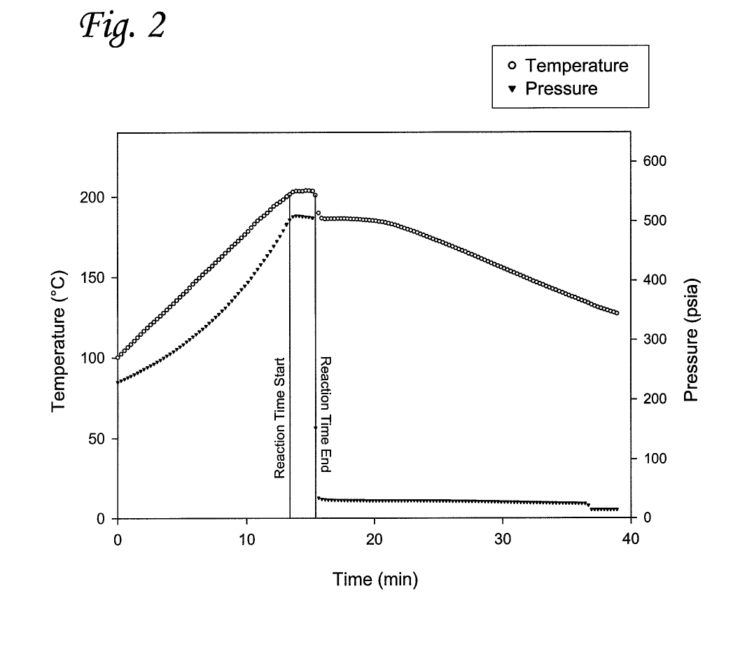

A series of 25 experiments were performed using three variables: reaction time (2 minutes, 5 minutes, and 8 minutes), reaction temperature (200° C., 215° C., 230° C.) and reaction pressure (range of 315-700 psia). In each experiment, Tifton 85 bermudagrass (T85, 15 g) was immersed in 1450 mL of deionized water for a final solids concentration of 1% w/v. The sample was placed in an apparatus as depicted in FIG. 1. Table 1 depicts both the set values for temperature and pressure and the actual values obtained during the reactor runs. FIG. 2 shows the temperature and pressure profile during a typical run. The vessel was heated from ambient temperature to 100° C. in 8 minutes, at which point data collection began, and from 100° C. to a set point (e.g., 200° C.) in another 15 minutes. The temperature increased linearly to the set point, and then commonly exceeded the set point by 2-3° C., before decreasing slightly during the reaction time. The release of hot water reduced the pressure immediately to less than 50 psia, but the temperature of the grass remaining in the vessel typically decreased only 10-15° C. immediately, and then slowly over 20-30 minutes to 100° C. In general, the actual temperature deviated less than 5° C. from the targeted temperature (mean deviation was 2.6° C.), while the actual pressure generally deviated less than 40 psia from the targeted pressure (19 psia mean deviation). The rapid heating (6.67° C./min average) and cooling (4° C./min average) of the reactor justifies using the two minute temperature plateau region as the reaction time.

Four hydrolysis-dependent variables were examined: glucose dissolved in the hydrolysate, xylose dissolved in the hydrolysate, reducing sugars dissolved in the hydrolysate, and digestibility of the biomass. Of the four hydrolysis-dependent variables studied, three quantified the affect of the physical parameters of the reactor (time, temperature, and pressure) on the dissolution of simple sugars in liquid hydrolysate.

The mass of glucose dissolved over the tested ranges of temperature and time did not correlate with either of these two factors.

The mass of xylose and the total mass of reducing sugar both correlated linearly with the time and temperature, increasing as either parameter increased, but with the temperature having a slightly greater effect. The mass of xylose dissolved was determined to be described by the following model (33 degrees of freedom, F-test=29.67, R²=0.657, P<0.0001):

Xylose dissolved (mg)=62.6+(25.63×time)+(50.76×temperature)

In this model, both the time (−1=2 min, 0=5 min, +1=8 min) and temperature (−1=200° C., 0=215° C.; +1=230° C.) are represented as coded variables. Similarly, the mass of reducing sugar was described by the following coded model (35 degrees of freedom, F-test=26.96, R²=0.620, P<0.0001):

Reducing sugar dissolved (mg)=1225.0+(312.6×time)+(418.3×temperature)

The digestibility of the solid grass also correlated with the time and temperature. Digestibility was calculated by determining the sugar yield. Sugar yield involves PBHW treatment of biomass, followed by additional enzymatic hydrolysis of the PBHW-treated solid biomass. Sugar yield is defined herein as the mass of reducing sugar hydrolyzed per mass of sample by the post-PBHW treatment enzymatic hydrolysis. Both temperature and time during the PBHW treatment significantly affected this sugar yield. Specifically, the sugar yield was described by a (coded) model which included a quadratic term and an interaction term (24 degrees of freedom, F-test=27.81, R²=0.848, P<0.0001):

Sugar yield (mg/mg)=0.4074+(0.0304×time)+(0.0896×temperature)−(0.0448×temperature)−(0.0442×temperature×time)

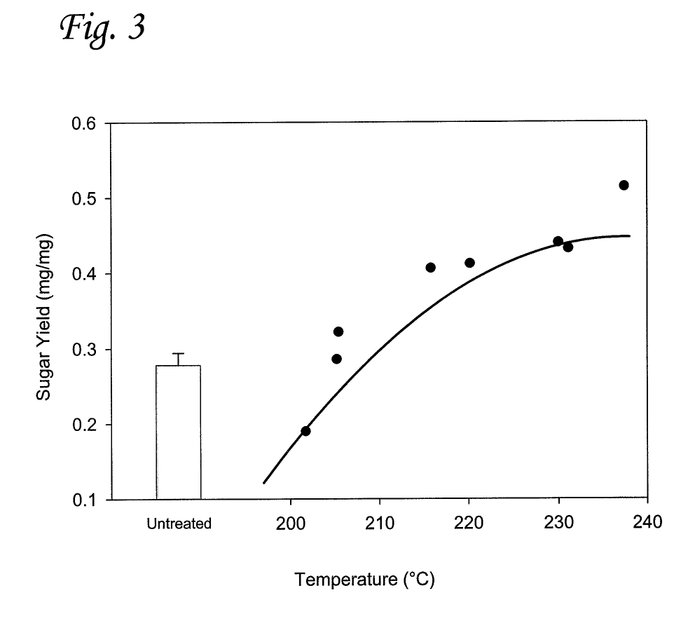

Although sugar yield increased linearly with both time and temperature, the presence of the negative interaction term caused the optimal time to be lower the greater the temperature. Moreover, the maximum sugar yield within the range studied occurred at the highest temperature (230° C.) and lowest time (2 min.), while the minimum occurred at the lowest temperature (200° C.) and time (2 min.). This phenomenon for a two minute hydrolysis time is shown in FIG. 3.

By combining net weight loss data with the NIR data, the percent dissolution of cellulose, hemicellulose, and lignin were estimated. There is a significant increase in the dissolution of hemicellulose for the 230° C. pretreatment (54%) over the 200° C. pretreatment (21%). A modest increase in cellulose dissolution from 6% at 200° C. to 11% at 230° C. was observed. Lignin dissolution increased from 0% to 5% at the higher temperature.

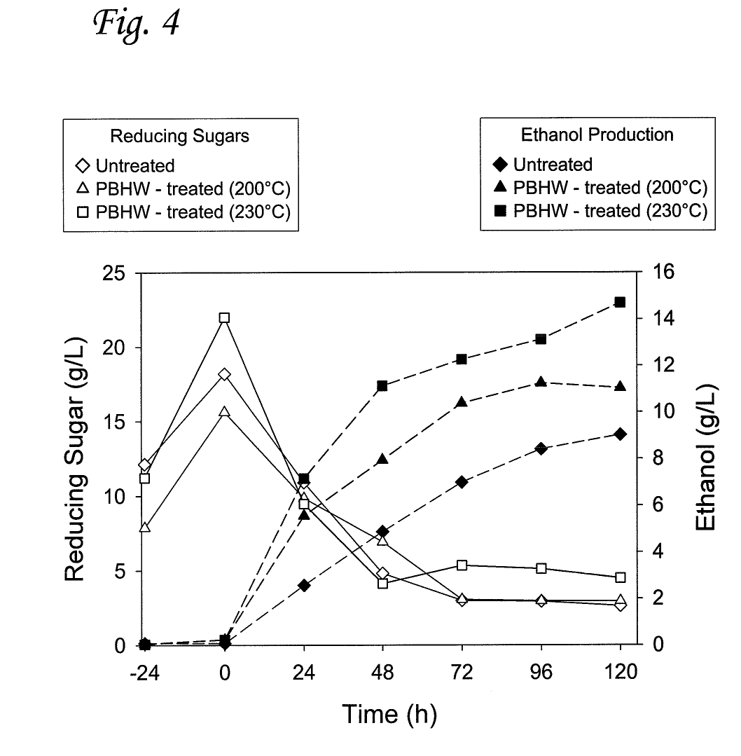

To confirm that increase in digestibility would correlate in increased ethanol yield, a series of partial saccharification and co-fermentation experiments (PSCF) were conducted using three conditions: untreated T85, 200° C. (2 min.) treated T85, and 230° C. (2 min.) treated T85. There was no furfural or 5-hydroxymethylfurfural (5-HMF) present in the hydrolysate following the pretreatments (data not shown). Table 2 outlines the profile of sugars released by the PBHW pretreatments as well as by the 24 hour enzymatic hydrolysis. Minimal sugars were released by the PBHW pretreatment alone. More arabinose and xylose were released from the 230° C. treated solids than the 200° C. or untreated solids which corresponded well with the increased dissolution of hemicellulose that occurred in the 230° C. treated grass (Table 2). At time zero there is more glucose liberated in the untreated grass, perhaps because autoclaving liberated the easily released sugars and these sugars had already been released in the PBHW pretreated samples. However, after 24 hours of enzymatic hydrolysis, the glucose released from either pretreatment of the grass solids is very similar and higher than the untreated grass, presumably due to enhanced accessibility of the cellulose. Ethanol production and reducing sugar levels over the course of the fermentations are shown in FIG. 4. As expected from the preliminary reducing sugar analyses, the 230° C. pretreated grass resulted in an increase in ethanol production of roughly 4.5 g/L over the 200° C. pretreated grass. Untreated grass produced the least amount of ethanol of the experiment (9 g/L).

Phenolic acids, p-coumaric acid and ferulic acid, were also released during the fermentations. There were small amounts of these compounds in the hydrolysate from each of the pretreatment conditions. Following enzyme addition and inoculation of the fermentations, both p-coumaric and ferulic acid levels increased over the 120 hours. Of the three conditions, the levels of both compounds were highest in the untreated grass. Hydrolysate samples from the PBHW pretreatment were also analyzed for furfural and 5-HMF, neither of which was present.

PBHW pretreatment is promising for increasing the digestibility of complex carbohydrates in plant biomass for subsequent processing and/or treatments. Our non-flow-through PBHW reactor is reliable and effective; pressure and temperature were held constant over the reaction time and significant dissolution of complex carbohydrates occurred as measured by enzymatic hydrolysis. The first objective was to evaluate the effectiveness of enzymatic hydrolysis of PBHW-pretreated T85 bermudagrass compared to untreated grass samples. Cellulase and cellobiase enzymes used for this aspect of our studies have been used previously to determine the effectiveness of cellulose degradation from pretreated biomass. (Foster et al., Appl. Biochem. Biotechnol., 91-3:269-282, 2001; Dale et al., Bioresour. Technol., 56:111-116, 1996; Holtzapple et al., Appl. Biochem. Biotechnol., 28-29:59-74, 1991). The later enzymatic hydrolysis reactions and subsequent fermentations were conducted in order to correlate digestibility with fermentability of PBHW-pretreated bermudagrass. Several different commercial enzyme combinations were compared for their ability to liberate sugars from cellulose and hemicellulose. Although all performed well, the Novozyme batch preparations used during the fermentation study performed the best for our current protocol.

Once high pressure conditions (e.g., greater than about 200 psia) are established for PBHW treatment, subsequent variations in pressure had a negligible affect on sugar yield from T85. Even though heating and cooling the sample took longer than the specified reaction time, the rates of heating and cooling were rapid enough that the plateau region (see, FIG. 2) is justified as the actual reaction time. After determining the sugar yield of the reactions in Table 1, the data was fit to a graph (FIG. 3). Based on the data presented in FIG. 3, we determined the optimal reaction time of 2 minutes and temperature of 230° C. We conducted the pretreatment at 1% w/v solids concentration since low solids concentrations may also be critical to effective pressurized batch hot water pretreatment. (Laser et al., Bioresour. Technol., 81:33-44, 2002).

An advantage of this pretreatment is that it does not require the use of a strong base or acid, as are in ammonia fiber explosion (AFEX) and dilute acid hydrolysis (DAH) pretreatments, respectively. Not only does this remove the additional cost of these reagents, but it eliminates the expense for their subsequent safe removal and disposal.

Inhibitors are often produced by biomass degradation during pretreatment and hydrolysis steps and include, for example, phenolics from lignin degradation and furfural and 5-HMF produced when monomeric sugars are degraded into aldehydes or reactive acids. One study found that these are produced by liquid hot water (LHW) pretreatment when O-acetyl and uronic groups from hemicellulose are cleaved and become reactive acids. (Mosier et al., Bioresour. Technol., 96:673-686, 2005) The high temperatures and pressures in certain LHW pretreatments accelerate this acid-catalyzed degradation of monomeric sugars by decreasing the pH as organic acids are formed. This is a result of the pretreatment as well as the substrate that is being treated. Weil and colleagues found that controlling the pH of yellow poplar wood sawdust, which reached a pH between 2.8 and 3, during a LHW pretreatment by adding base prevented the formation of inhibitors. The pH of the liquid hydrolysate from our reactor ranged from 4.2 to 4.8 and may not have been low enough to promote significant formation of inhibitors. The short residence time of the pretreatment likely prevented the formation of inhibitors as well. The absence of these compounds in this study is promising for future applications of our PBHW system.

Samples after pretreatment, at the beginning, and at the end of the fermentations were also analyzed for phenolic acids, specifically p-coumaric and ferulic. These compounds are released from grasses during hydrolysis and are inhibitory to fermentations. Ferulic acid and its related compounds possess potent antioxidant properties and may have applications in disease prevention and treatment. Extraction of these compounds prior to fermentation could be pursued further and may serve as a potential source of value-added by-product from ethanol production in addition to increasing ethanol yields.

PBHW is an effective and gentle pretreatment resulting in greater enzymatic digestibility of T85 bermudagrass. For our reactor, 230° C. is the most efficient temperature for increasing the digestibility without producing detrimental concentrations of inhibitors. The increased digestibility directly resulted in an increased ethanol yield from fermentations using E. coli LY01. The results of this study warrant further research to determine the efficacy PBHW pretreatment for other biomass sources and possibly use on a larger scale.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Pressurized Batch Hot Water (PBHW) Hydrolysis Reaction

Tifton 85 bermudagrass obtained from the USDA-ARS Coastal Plain Experiment Station (Tifton, Ga.) was used for all hydrolysis studies. The bermudagrass was harvested at four weeks and dried in the field in bales for an additional week. The moisture content of the grass was determined to be 6.5% by drying at 110° C. for 1 hour.

PBHW hydrolysis was examined in a 2-liter pressure vessel (Model 4600 Parr Instrument Co., Moline, Ill.) surrounded by retractable ceramic heaters (FIG. 1). Approximately 15 grams (g) of unprocessed grass was placed in a 500 micron (μm) (35 mesh) stainless steel basket and then immersed in 1450 millilters (mL) of deionized water in the vessel for a final solids concentration of 1% w/v.

Prior to reaction cycles, the vessel head plate was secured and the headspace purged with nitrogen via two ports. The reaction cycle began by heating the vessel to a set point temperature. The vessel was filled with nitrogen at room temperature to achieve a target pressure at the set point temperature. Reaction cycles were performed as shown in Table 1.

Heating, release of vessel contents, and collection of reaction time, reaction temperature, and reaction pressure data were measured via a datalogger and associated software (Model 21X micrologger, Campbell Scientific, Inc., Logan Utah). “Reaction time” was the time set to elapse from the moment the contents of the reactor first reached the set point temperature to the moment the outlet valve automatically opened. “Reaction temperature” and “reaction pressure” were calculated as the mean of each variable recorded at 15 second intervals during the reaction time. After the reaction time elapsed at the set point temperature, an 80 pound per square inch (psi) pneumatically-actuated ball valve released the liquid hydrolysate from the pressure vessel to a partially evacuated condenser cooled by tap water. The hot liquid was cooled to less than 50° C. and the system depressurized to less than 40 psi in roughly ten seconds. The hydrolyzed solids (wet but no longer pressurized) remained in the basket to cool. As a safety precaution, a low pressure switch at the water inlet required a minimum pressure of 10 psi to actuate the pneumatic valve and to allow the ball valve to release the hydrolysate into the condenser. A manual ball valve to release the condensate and a 50 psi pressure relief valve were located at the outlet of the condenser. The hydrolyzed solids were then removed from the vessel and dried at 40° C. for 90 minutes (min) using a fluidized bed dryer (Endecott FBD2000, London, UK). Liquid and dried samples were stored (at −20° C. and 4° C., respectively) for subsequent enzyme and fermentation studies.

Temperature was monitored inside the vessel using two 1.5 millimeter (mm) platinum resistance temperature detectors (RTDs, Model PR11, Omega Engineering, Inc, Stamford, Conn.). One RTD was connected to the process controller (CN8200 Series, Omega Engineering, Inc., Stamford, Conn.) for the system. The remaining RTD was connected to the datalogger. A pressure transducer (PX02 Series, Omegadyne, Inc., Sunbury, Ohio) occupied a port on the reactor head plate. The vessel, valves, and sensors were designed to withstand operating conditions of 350° C. and 1000 psi, and the maximum operating conditions used in this study were 230° C. and 700 psi (5 MPa).

### Post-PBHW Enzymatic Hydrolysis

After PBHW pretreatment, dried bermudagrass samples were ground in a mill (Cyclotech Model Sample mill, Foss, Tecator, AB Hognas, Sweden) and further hydrolyzed enzymatically for later statistical analysis. The enzyme reaction was conducted for 48 hours at 40° C. in a 0.05 M citrate buffer solution pH 4.5 with a 5% (w/v) solids load. Sodium azide was added at 0.15% (w/v) to inhibit microbial contamination. Celluclast 1.5 FG containing approximately 102 filter paper units (FPU)/mL and Novozyme 431 containing approximately 250 cellobiase units (CBU)/mL (both from Novozymes, Franklinton, N.C.) were loaded at a rate of 4.5 FPU and 44.3 CBU per gram of dry weight of bermudagrass. Samples were boiled for 15 minutes to terminate enzymatic hydrolysis.

TABLE 1

| Set | Reaction | | Initial | | Reaction |
|---|---|---|---|---|---|
| Temperature | temperature | Time | Pressure | Set pressure | Pressure |
| (° C.) | (° C.) | (min) | (psig) | (psig) | (psig) |
| 200 | 205.4 | 2 | 35 | 315 | 352.7 |
| 200 | 205.2 | 2 | 80 | 400 | 436.5 |
| 200 | 201.7 | 2 | 140 | 525 | 519.6 |
| 200 | 206.1 | 5 | 35 | 315 | 342 |
| 200 | 201.8 | 5 | 140 | 525 | 532.9 |
| 200 | 202.3 | 8 | 35 | 315 | 310.9 |
| 200 | 202.1 | 8 | 80 | 400 | 415.4 |
| 200 | 201.1 | 8 | 140 | 525 | 522 |
| 215 | 215.8 | 2 | 37 | 400 | 372.5 |
| 215 | 216.3 | 2 | 140 | 625 | 620.8 |
| 215 | 215.7 | 5 | 105 | 550 | 548.9 |
| 215 | 224.9 | 5 | 105 | 550 | 610.9 |
| 215 | 216.9 | 5 | 105 | 550 | 548.4 |
| 215 | 215.4 | 5 | 105 | 550 | 544.4 |
| 215 | 216.1 | 5 | 105 | 550 | 534 |
| 215 | 212.1 | 8 | 37 | 400 | 378.4 |
| 215 | 215.5 | 8 | 140 | 625 | 627.8 |
| 230 | 231.1 | 2 | 0 | 415 | 418.2 |
| 230 | 231.7 | 2 | 57 | 550 | 560 |
| 230 | 230 | 2 | 120 | 700 | 696.8 |
| 230 | 231.2 | 5 | 0 | 415 | 409.5 |
| 230 | 230.2 | 5 | 120 | 700 | 644.7 |
| 230 | 229.1 | 8 | 0 | 415 | 408.5 |
| 230 | 230.4 | 8 | 57 | 550 | 526.8 |
| 230 | 230.5 | 8 | 120 | 700 | 682.2 |

TABLE 2

| | Arabinose (mg/g grass) | | | Xylose (mg/g grass) | | | Glucose (mg/g grass) | | |
|---|---|---|---|---|---|---|---|---|---|
| Treatment | Hydrolysate | 0 hrs | 24 hrs | Hydrolysate | 0 hrs | 24 hrs | Hydrolysate | 0 hrs | 24 hrs |
| Untreated | n/a | 48.91 | 60.45 | n/a | 14.79 | 29.88 | n/a | 371.69 | 541.63 |
| 200° C. Treated | 0 | 56.28 | 69.23 | 0 | 60.03 | 133.15 | 0.69 | 244.09 | 640.34 |
| 230° C. Treated | 0.25 | 79.39 | 76.75 | 0 | 129.49 | 282.36 | 0.31 | 209.5 | 635.32 |

### Statistical Analysis of Enzymatic Hydrolysis

Concentrations of reducing sugars in the hot water hydrolysate and the enzyme hydrolysate were measured using the dinitrosalicylic acid assay (Miller, G. L., Anal Chem., 31(3):426-428, 1959) with glucose as the standard. Glucose and xylose concentrations of the hot water hydrolysate were determined by HPLC. These values were then applied to a Box-Behnken response surface statistical design (Design-Expert software, Stat-Ease, Inc., Minneapolis, Minn., USA) to evaluate the performance of the pressurized water vessel and the effect of temperature (targeted as 200-230° C.), pressure (targeted to be in range 315-700 pound per square inch absolute (psia)) and reaction time (2-8 minutes) on four hydrolysis dependent variables: glucose dissolution, xylose dissolution, total reducing sugars dissolution, and the enzymatic digestibility of the remaining solid material. The actual recorded reaction temperature and pressure, rather than the target values, were used in the statistical analysis. The saturated model was fit for each of these variables according to the constraints of the design (the model with linear terms, two-way interaction effects plus quadratic effects), and eliminated the non-significant terms to yield a reduced model. The values for the pressure were constrained by the vapor pressure of water at the reaction temperature (minimum) and by the permissible pressure in the condenser (maximum), therefore additional values for this parameter were used in the statistical analysis. Results are shown in FIG. 3.

### PBHW Hydrolysis for Fermentation

T85 bermudagrass was treated by PBHW at 200° C. and 230° C. at 1% w/v solids to generate enough material for two fermentations of each treatment. The hydrolysate collected was analyzed for sugars, furfural, 5-hydroxymethylfurfural (5-HMF), p-coumaric acid, and ferulic acid using HPLC. Treated grass was dried as described previously. Following drying, grass samples with the same treatment were combined, ground twice in a Fritsch Pulverisette 25 (6.0 grill) (Laval Labs, Laval (Quebec) Canada), and ground in a coffee grinder (IDS77 Mr. Coffee, Inc., Bedford Heights, Ohio). Final particle size varied between 0.1 mm to 3 mm. Percent moisture was determined by drying a sample of each condition overnight in a drying oven at 100° C. Grass samples were then analyzed for neutral detergent fiber (NDF), acid detergent fiber (ADF), lignin (ADL), and protein NIR at the Feed and Environmental Water Lab (FEW-AESL, University of Georgia, Athens, Ga.), according to standard protocols.

### Fermentations

Grass and dH₂O were added to obtain 10% w/v solids in 100 mL and autoclaved. Subsequently, 190 mL of 2× Luria Bertani medium (LB, Fisher, Fair Lawn, N.J.) was added. Novozymes (Franklinton, N.C.) Batch NS50012 (23 FPU/mL, 443 IU/mL xylanase, and 3497 Polygalacturonase units (PGU)/mL) and Batch NS50013 (57 FPU/mL, 4049 IU/mL xylanase, and 12 PGU/mL) were filter sterilized, added to 2×LB, and then added to the fermentors for a final concentration of 2 FPU/g dry wt substrate. The pH was adjusted to 4.5 with 2N HCl. These mixtures were incubated in a 45° C. circulating water bath with stirring for 22 hours.

Escherichia coli strain LY01 was inoculated from glycerol stocks and incubated at 37° C. for 18 hours in LB containing 50 g glucose and 40 mg chloramphenicol. Fermentors were inoculated for a starting OD₅₅₀ of 1. The pH was adjusted to 5.5 with KOH, and water temperature bath decreased to 35° C.

Samples were taken every 24 hours for 120 hours. Samples were filtered (Corning SPIN-X Centrifuge Tube Filter 0.22 μm, Sigma-Aldrich, St. Louis, Mo.), stored in O-ring microfuge tubes, and frozen at −80° C. Reducing sugars were determined as described (Miller, Anal. Chem., 31:426-428, 1959). Filtered samples were analyzed for ethanol by gas chromatography (Shimadzu GC-8A, Columbia, Md.) as previously described in Doran J et al., Appl. Biochem. Biotechnol., 84:141-152 (2000) using a flame ionization detector and the parameters: injector/detector temperature of 250° C., column temperature of 65° C., 0.53 mm ID×30 m column with 3 μm film. Samples were also analyzed for phenolic acids by HPLC and for sugars by gas chromatography. Results are shown in FIG. 4.

### Analysis of Soluble Carbohydrates

25 μL of filtered liquid sample was blown to dryness by nitrogen after adding 50 μL of MeOH containing 91 μg of phenyl glucose as the internal standard. One-two drops of acetonitrile were also added to dried samples and then blown to dryness again. Silylation was performed by adding 50 μL of both trimethylsilane (TMS) and N, O-Bis (trimethylsilyl) trifluoroacetamide (BSTFA) to dried samples followed by incubation at 75° C. for 30 minutes. Arabinose, xylose, and glucose, both α and β conformations, were determined for 1 μL aliquots of silylated sugar derivatives by gas chromatography (model 5890, Hewlett Packard Inc., Atlanta, Ga.) using J&W DB-5 capillary column (30 M×0.25 mm I.D.) (Agilent, Wilmington, Del.). The temperature program started at 155° C., and increased to 215° C. at a rate of 1.3° C./min. The temperature then increased to a final temperature of 320° C. at a rate of 5° C./min. Injector temperature was 250° C. and detector temperature was 350° C.

### Phenolic Quantification

This procedure was adapted from a chlorogenic acid quantification protocol. 100 μL of sample was diluted with 100 μL dH₂O. 50 μL of MeOH containing 0.0403 mg of chrysin was added as an internal standard. 3-(4-hydroxy-3-methoxy-phenyl)prop-2-enoic acid (ferulic) and 3-(4-hydroxyphenyl)-2-propenoic (p-coumaric) acid concentrations were determined for 20 μL aliquots of the solution by reverse-phase HPLC (model 1050, Hewlett Packard Inc., Atlanta, Ga.) using an H₂O/MeOH linear gradient from 10% to 100% MeOH in 35 minutes and a flow rate of 1 mL/min. The column was a 250 mm×4.6 mm ID, 5 μm Ultrasphere C18 (Beckmann Instruments Inc., Norcross, Ga.). The detector was a diode array system and 340 nm was used for further analysis. Each solvent contained 0.1% H₃PO₄. Response factors were determined with pure authentic compounds (Sigma-Aldrich Co., St. Louis, Mo.). Quantification of ferulic and p-coumaric acid was based on the internal standard (chrysin) and peak identification was based on co-chromatography (spiking) and spectral analysis.

### Example 2

Sorghum (5% w/v solids) (Coffey Forage Seeds; Inc., Plainview, Tex.) was treated in the pressurized batch hot water reactor as generally described in Example I, with treatment parameters of heating to 230° C. for two minutes at an initial pressure of 57 psig. Liquid was collected in the process. Fermentations were then carried out on the untreated (UN) and the pressurized batch hot water (PBHW)-treated samples to evaluate the maximum ethanol yield. UN and PBHW-treated samples were either treated with dilute acid (DAH) or untreated (dH₂O) before fermentation with E. coli strain LY40A (U.S. Provisional Patent Application Ser. No. 61/097,975, filed Sep. 18, 2008). Distilled H₂O was used in the untreated samples instead of 0.88% w/v H₂SO₄ used in treated samples. Fermentations were also carried out on the PBHW-treated sample mixed with liquid that was collected during the PBHW treatment process.

### Moisture Content

Untreated sample: 9.63%

PBHW-treated sample: 11.24%

### Preparation of Samples:

The untreated (UN) samples were prepared by adding 8.5 ml of either 0.88% w/v H₂SO₄ (½ DAH) or distilled water (dH₂O) to 1.5 g dry weight (1.66 g wet wt) of untreated (UN) sample weighed in a 50 mL screw cap glass. The PBHW-treated samples were prepared by adding 8.5 mL of either 0.88% w/v H₂SO₄ (½ DAH), liquid from treatment process (Liq), or distilled water (dH₂O) to 1.5 g dry weight (1.69 g wet wt) of PBHW-treated sample weighed in a 50 mL screw cap glass centrifuge tube.

The sample mixtures were autoclaved for 1 hour at 121° C.

E. coli LY40A Fermentations

After autoclaving, the sample mixtures were allowed to cool and 1.5 mL of 10×LB media were added. 10% w/v Ca(OH)₂ and 1 M citric acid were used to adjust the pH to 4.5. Prior to inoculating and incubating, enzymatic pretreatment of the sample mixtures was performed by adding 90 μL cellobiase (final concentration, 60 U/g of substrate) and 450 μL Novozyme NS50013 (final concentration, 15 FPU/g of substrate). Each of the cellobiase and Novozymes NS50013 were obtained from Novozymes, Franklinton, N.C. All sample mixtures were incubated for 24 hours at 45° C. under shaking conditions.

E. Coli LY40A was transferred from −80° C. to Luria Agar (LA, Fisher, Fair Lawn, N.J.) plates containing 2% glucose and 60 mg chloramphenicol (CAM). After 24 hours of incubation, a single colony was inoculated in LB broth with 5% glucose and 40 mg CAM and incubated at 37° C. overnight. The grown culture was then centrifuged, re-suspended in LB and added to each sample mixture to a final O.D. of 1 at A₅₅₀ per tube. The total volume was adjusted to 15 mL and the tubes were incubated at 37° C. under shaking conditions and pH was adjusted to 5.5. Samples were taken every 24 hours to estimate ethanol by gas chromatography. Results are shown in Tables 3 and 4.

TABLE 3
Untreated samples (UN)

Ethanol produced (g/L)

| Time | dH₂O | dH₂O | ½DAH | ½DAH |
|---|---|---|---|---|
| (h) | 1 | 2 | 1 | 2 |
| 24 | 12.13 | 14.82 | 0.29 | 5.18 |
| 48 | 11.87 | 15.82 | 12.59 | 13.95 |
| 72 | 9.09 | 14.31 | 11.54 | 14.86 |

½ DAH = dilute acid treated
dH₂O = Distilled water

TABLE 4
PBHW treated sample

| | Ethanol produced (g/L) | | | | | |
|---|---|---|---|---|---|---|
| | dH₂O | dH₂O | ½DAH | ½DAH | | |
| Time (h) | 1 | 2 | 1 | 2 | Liq. 1 | Liq. 2 |
| 24 | 18.31 | 14.67 | 0.13 | 0.22 | 8.37 | 23.04 |
| 48 | 21.02 | 22.06 | 12.77 | 14.43 | 22.59 | 21.01 |
| 72 | 20.59 | 21.91 | 11.83 | 14.92 | 23.84 | 20.65 |

½ DAH = dilute acid treated
dH₂O = Distilled water
Liq = liquid from PBHW treatment process

### Example 3

50 g of sweet sorghum (Coffey Forage Seeds; Inc., Plainview, Tex.) in a total volume of 1 L (5% w/v solids) was treated in the pressurized batch hot water reactor as generally described in Example I, with treatment parameters of heating to 230° C. for 2 minutes at an initial pressure of 57 psig. The hydrolysate was collected and stored for later use.

Moisture content:

Untreated: 10.54%

PBHW-treated: 19.1%

Pretreated to receive hydrolysate: 13.33%

Samples were dried and ground in a Wiley Mill (Thomas Scientific, Swedesboro, N.J.) [with a 2 mm screen prior to fermentation.

### Fermentations

Two fleakers per loading were used, for a total of six fleakers. 10% dry weight of each sample of sorghum was used per fleaker. Distilled water was added to each fleaker to final volume of 100 mL. After autoclaving and cooling, 80 mL of 2×LB was added to each fleaker along with filter sterilized enzymes in 10 mL of 2×LB: 15 FPU/g dry weight of Novozyme NS 50013 (Novozymes, Franklinton, N.C.) and 60 CBU/g dry weight of Novozyme Novo 188 (Novozymes, Franklinton, N.C.). The enzymatic preincubation was run for 24 hours at 45° C. with an initial pH of 4.5.

E. coli LY40A was transferred from −80° C. to LB plates containing 2% glucose and 40 mg chloramphenicol (CAM). After 24 hours of incubation, a single colony was inoculated in 100 mL of LB and allowed to grow for 24 hours. The grown culture was then concentrated to A₅₅₀=50 in 2×LB and added to a final OD of 0.5 at A₅₅₀ per fleaker. After inoculation the temperature was adjusted to 35° C. and the pH maintained at 5.5. The fermentations were run at these conditions for 120 hours. Results are shown in Tables 5 and 6.

TABLE 5
Average Ethanol yield from sweet sorghum (g/L)

| | | | PBHW + |
|---|---|---|---|
| Time | Untreated | PBHW-treated | Hydrolysate |
| −24 | 0 | 0 | 0 |
| 0 | 0 | 0 | 0 |
| 24 | 1.56 | 5.26 | 0.28 |
| 48 | 3.91 | 10.65 | 4.99 |
| 72 | 5.28 | 11.63 | 8.09 |
| 96 | 6.85 | 11.98 | 8.93 |
| 120 | 6.33 | 11.99 | 9.53 |

TABLE 6
Average sugar in fermentation

| Time | PBHW-treated | Untreated | PBHW + hydrolysate |
|---|---|---|---|
| −24 | 10.01674 | 19.59681 | 18.34733 |
| 0 | 30.23017 | 42.74869 | 44.21882 |
| 24 | 18.07282 | 22.83074 | 35.39899 |
| 48 | 22.67001 | 9.821504 | 18.64133 |
| 72 | 9.943503 | 6.955061 | 9.160017 |
| 96 | 10.67587 | 6.440571 | 8.645527 |
| 120 | 12.21385 | 3.50063 | 7.690046 |

### Example 4

Cotton (Gossypium spp.) (5% w/v solids) is pretreated as generally described in Example I, with treatment parameters of heating to 230° C. for two minutes at an initial pressure of 57 psig. A second sample of cotton is pretreated as generally described in Example I, with treatment parameters of heating to 240° C. for two minutes at an initial pressure of 57 psig. Hydrolysate is collected from each sample.

Cotton contains a relatively higher ratio of cellulose versus hemicellulose compared to grassy substrates. Cellulose can be more resistant to disruption than hemicellulose, thus the higher temperature in the second sample can result in more complete disruption of the cotton substrate. The reaction time is maintained at two minutes to limit the extent to which sugars released into the hydrolysate are degraded.

### Example 5

Woody plant biomass such as pine (Pinus spp.) or poplar (Populus spp.) is reduced to particle size between chips and sawdust. The sample (5% w/v solids) is treated as generally described in Example I, with treatment parameters of heating to 230° C. for two minutes at an initial pressure of 57 psig. A second sample is treated as generally described in Example I, with treatment parameters of heating to 240° C. for two minutes at an initial pressure of 57 psig. A third sample is treated as generally described in Example I, with treatment parameters of heating to 230° C. for five minutes at an initial pressure of 57 psig. Hydrolysate is collected from each sample.

Woody plant biomass can be more recalcitrant than grassy biomass substrates. Thus, the somewhat more severe reaction conditions used in the pretreatment of the second and third samples may result in more disruption of the substrate.

### Example 6

Tifton 85 bermudagrass (USDA—ARS Coastal Plain Experiment Station, Tifton, Ga.) was pretreated as generally described in Example I, with treatment parameters of heating to 230° C. for 2 minutes at an initial pressure of 57 psig. An untreated sample of Tifton 85 bermudagrass was provided as a control.

Hypocrea jecorina (ARS Culture Collection, NCAUR, Peoria, Ill.) was incubated in the presence of pretreated or untreated grass (2 g) in 30 mL of the following media: 15.0 g KH₂PO₄, 20.0 g corn steep liquor (Sigma-Aldrich, St. Louis, Mo.), 5 g NH₄SO₄, 0.5 g Mg(SO₄)₂ 7H₂O, and 1.0 mL Tween 80, all in one liter. Basal medium was adjusted to pH 4.8 with 1 MNaOH. The fungus was grown at 28° C. with agitation (250 rpm) for 8 days.

Catalytic activity produced by H. jecorina after incubation with the pretreated and untreated substrates was analyzed. Enzyme activities in the presence of 1% w/v substrates (Sigma-Aldrich, St. Louis, Mo.) including oat-spelt xylan, polygalacturonic acid, carboxymethylcellulose (CMC, low viscosity), 15 mmol/L cellobiose, and 2.5 mmol/L p-nitrophenyl (p-NP) conjugated substrates were determined at 50° C. in the presence of 50 mmol/L sodium acetate buffer, pH 4.8 using published methods (Ximenes et al., Applied Biochemistry Biotechnology, 137-140:171-183, 2007; Berlin, A. et al., Applied Biochemistry Biotechnology, 121-124:163-170, 2005). Filter paper activity (FPAase) was assayed as described by Mandels et al., Biotechnology Bioengineering, 6:17-34, 1976). Release of reducing sugars was determined according to Miller, Analytical Chemistry, 31:426-428, 1959). Glucose determination for the cellobiase assay used a D-glucose (GOPOD Format) assay kit from Megazyme (Megazyme International Ireland Ltd., Co. Wicklow, Ireland). One unit of cellulase (for CMC as substrate), cellobiase, xylanase and polygalacturonase activities was defined as the release of one μmol of glucose, xylose or galacturonic acid respectively, per min. For p-NP conjugated substrates, one unit of activity was defined as one μmol of p-NP released per min.

Detected cellulolytic activities included filter paper activity (FPAase), carboxymethyl cellulose (CMCase), β-glucosidase (β-GL), and cellobiase and are reported in Table 7. Detected hemicellulolytic activities included xylanase, β-xylosidase (β-xyl), α-arabinofuranosidase (α-arf), α-galactosidase (α-gal), polygalacturonase (PG), and amylase and are reported in Table 8.

TABLE 7

| | FPAase | CMCase | β-GL | Cellobiase |
|---|---|---|---|---|
| Sample | (FPU) | (IU/mL) | (IU/mL) | (CBU) |
| Untreated | 0.53 | 14.8 | 0.21 | 0.25 |
| Treated | 1.16 | 11.5 | 0.12 | 0.17 |

TABLE 8

| | Xylanase | β-xyl | α-arf | α-gal | PG | Amylase |
|---|---|---|---|---|---|---|
| Sample | (IU/mL) | (IU/mL) | (IU/mL) | (IU/mL) | (IU/mL) | (IU/mL) |
| Untreated | 85.0 | 0.53 | 0.56 | 0.60 | 0.31 | 0.60 |
| Treated | 32.2 | 0.70 | 0.28 | 0.15 | 0.00 | 0.19 |

An enzyme preparation from H. jecorina was prepared by growing H. jecorina (ARS Culture Collection, NCAUR, Peoria, Ill.) on untreated Tifton 85 bermudagrass for eight days at 28° C., pH 4.8 and collecting the supernatant.

The H. jecorina enzyme preparation was compared to commercially available enzyme preparations for hydrolysis of treated and untreated Tifton 85 bermudagrass substrates. Enzyme activities were determined as described above.

Table 9 shows results of the analysis of the indicated preparations standardized for xylanase activity (400 IU/g of substrate). Tested preparations included SPEZYME CP (Genecor International, Inc., Rochester, N.Y.) and DEPOL 740L (Biocatalysis Inc., Wales, United Kingdom). The values reported in Table 9 are provided as mg per gram of substrate.

TABLE 9

| | Reducing | | | Arabi- | Ferulic |
|---|---|---|---|---|---|
| Sample | sugars | Glucose | Xylose | nose | Acid |
| SPEZYME CP | 391.2 | 221.4 | 72.0 | 0.78 | 0.25 |
| H. jecorina prep | 282.0 | 132.8 | 64.0 | 0.77 | 0.26 |
| DEPOL 740L | 218.0 | 94.8 | 63.3 | 7.48 | 0.88 |
| SPEZYME CP + | 440.0 | 123.3 | 85.9 | 3.73 | 0.36 |
| DEPOL 740L | | | | | |
| H. jecorina prep + | 373.0 | 92.6 | 78.4 | 3.70 | 0.54 |
| DEPOL 740L | | | | | |

Table 10 shows the results of the analysis of preparations standardized for cellulose activity as follows: FPAase activity of SPEZYME CP and H. jecorina enzyme preparation were set for 8 FPU/g substrate, feuloyl esterase activity of DEPOL 740L was set to 7.8 IU/g substrate, and cellobiase activity was set to 78.2 CBU/g substrate. Tested preparations included SPEZYME CP (Genecor International, Inc., Rochester, N.Y.), DEPOL 740L (Biocatalysis Inc., Wales, United Kingdom), and cellobiase Novozymes 188 (Novozymes, Franklinton, N.C.). The values reported in Table 10 are provided as mg per gram of substrate.

TABLE 10

| | Reducing | | | Arabi- | Ferulic |
|---|---|---|---|---|---|
| Sample | Sugars | Glucose | Xylose | nose | Acid |
| SPEZYME CP | 321.0 | 218.1 | 60.5 | 0.45 | 0.17 |
| H. Jecorina prep | 316.0 | 180.9 | 51.2 | 1.27 | 0.21 |
| DEPOL 740L | 218.0 | 94.8 | 63.3 | 7.48 | 0.88 |
| SPEZYME CP + | 390.0 | 122.8 | 71.2 | 8.04 | 0.36 |
| DEPOL 740L | | | | | |
| H. Jecorina prep + | 338.8 | 115.9 | 89.2 | 1.85 | 0.39 |
| DEPOL 740L | | | | | |
| H. Jecorina prep + | 352.0 | 230.1 | 47.9 | 0.13 | 0.27 |
| cellobiase | | | | | |
| SPEZYME CP + | 364.0 | 306.8 | 53.0 | 0.15 | 0.12 |
| cellobiase | | | | | |
| SPEZYME CP + | 460.0 | 230.9 | 54.5 | 1.04 | 0.42 |
| DEPOL 740L + | | | | | |
| cellobiase | | | | | |

The complete disclosure of all patents, patent applications, and publications are incorporated by reference in their entirety as if each were incorporated by reference individually. In the event that any inconsistency exists between the disclosure of the present application and the disclosure of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. An apparatus comprising:
a vessel comprising:
at least one wall comprising:
an internal surface defining a chamber, and
an external surface comprising an edge defining an opening to the chamber,
a cover configured to sealably close the chamber opening,
an inlet port providing fluid communication between the chamber and a pressure source,
a reversibly openable pressure outlet in fluid communication with the chamber,
a temperature detector in thermal communication with the chamber and in communication with a display, and
a pressure detector in communication with the chamber and in communication with a display; and
a heating element in thermal communication with the external surface of the vessel wall.

2. The apparatus of claim 1 wherein the cover comprises one or more of: the pressure inlet, the pressure outlet, the temperature detector, and the pressure detector.

3. The apparatus of claim 1 wherein the vessel further comprises a fluid outlet in fluid communication with the chamber.

4. The apparatus of claim 3 wherein the fluid outlet provides fluid communication between the chamber and a condenser, wherein the condenser comprises a condenser fluid outlet.

5. The apparatus of claim 3 further comprising a process controller in communication with at least one of: the heating element, the pressure source, the pressure outlet, and the fluid outlet.

6. The apparatus of claim 1 further comprising a process controller in communication with at least one of: the heating element, the pressure source, and the pressure outlet.

7. The apparatus of claim 1 further comprising a data acquisition unit in communication with at least one of: the temperature detector and the pressure detector.

8. The apparatus of claim 1 further comprising a timer.

9. The apparatus of claim 8 wherein the timer is in communication with the data acquisition unit.

10. A method of processing plant biomass, the method comprising:
providing plant biomass comprising at least one of: cellulose and hemicellulose;
heating the plant biomass under conditions and for a time sufficient to at least partially depolymerize one or more of: the hemicellulose and the cellulose, thereby yielding processed plant biomass;
wherein the time is at least one minute; and
wherein the conditions comprise:
a volume of liquid medium so that the plant biomass is provided in an amount of at least about 0.01% w/v solids up to about 50% w/v solids;
a temperature of at least about 180° C.; and
pressure of at least about 200 psia.

11. The method of claim 10 wherein the plant biomass comprises agricultural residue, forestry residue, or waste stream residue.

12. The method of claim 11 wherein the agricultural residues comprises leaves, grass, corn stover, corn cob, sugar cane stalk, sugar cane bagasse, sorghum stalk, or sorghum bagasse.

13. The method of claim 12 wherein the grass comprises sorghum, timothy grass, napier grass, bermuda grass, switchgrass, or a hybrid.

14. The method of claim 10 wherein the plant biomass comprises high cellulose material.

15. The method of claim 14 wherein the plant biomass comprises cotton.

16. The method of claim 14 wherein the plant biomass comprises woody plant biomass.

17. The method of claim 10 wherein the biomass comprises high pectin biomass.

18. The method of claim 10 wherein the time is at least two minutes.

19. The method of claim 10 wherein the temperature is at least 200° C.

20. The method of claim 19 wherein the temperature is at least 230° C.

21. The method of claim 10 wherein the pressure is at least 300 psia.

22. The method of claim 10 wherein heating the plant biomass comprises releasing fermentable sugars.

23. The method of claim 10 wherein the plant biomass is provided in an amount of at least about 0.1% w/v solids up to about 15% w/v solids.

24. The method of claim 23 wherein the plant biomass is provided in an amount of at least about 1% w/v solids up to about 5% w/v solids.

25. The method of claim 10 wherein the conditions comprise heating the plant biomass to at least 230° C. for at least two minutes.

26. The methods of claim 10 wherein the conditions comprise a pH of at least 4.0.

27. The method of claim 26 wherein the pH of at least 4.0 is maintained in the absence of adding a base.

28. The method of claim 10 wherein the liquid medium comprises water.

27. The method of claim 10 wherein heating the plant biomass produces a hydrolysate and the method further comprises enzymatically hydrolyzing the collected hydrolysate.

28. The method of claim 10 further comprising cooling and depressurizing the processed plant biomass.

29. The method of claim 10 performed under conditions that limit production of at least one fermentation inhibitor.

30. The method of claim 29 wherein the fermentation inhibitor is ferulic acid, para-coumaric acid, furfural, or 5-hydroxylethyl furfural.

31. The method of claim 10 further comprising enzymatically hydrolyzing the processed plant biomass.

32. The method of claim 31 wherein enzymatically hydrolyzing the processed plant biomass comprises incubating the processed plant biomass with the enzymatic composition under conditions effective for the enzyme composition to convert at least one of hemicellulose and cellulose to monomeric sugars.

33. The method of claim 32 wherein the enzymatic composition comprises at least one enzyme of Hypocrea jecorina.

34. The method of claim 32 wherein the processed plant biomass and the enzymatic composition are incubated at a temperature of from 27° C. to 40° C.

35. The method of claim 32 wherein the processed plant biomass substrate and the enzymatic composition are incubated from 12 hours to 120 hours.

36. The method of claim 32 wherein the enzymatic composition comprises at least one enzyme of H. schweinitzii.

37. A method of pretreating plant biomass, the method comprising:
providing plant biomass comprising at least one of: cellulose and hemicellulose;
pretreating the plant biomass under conditions and for a time sufficient to at least partially depolymerize one or more of: the hemicellulose and the cellulose, thereby yielding pretreated plant biomass;
wherein the time is at least one minute; and
wherein the conditions comprise:
a volume of liquid medium so that the plant biomass is provided in an amount of at least about 0.01% w/v solids up to about 50% w/v solids;
a temperature of at least about 180° C.; and
pressure of at least about 200 psia; and
subjecting the pretreated plant biomass to additional treatment.

38. The method of claim 37 wherein the additional treatment comprises enzymatic hydrolysis.

39. The method of claim 37 wherein pretreating the plant biomass produces a hydrolysate; and
enzymatically hydrolyzing the collected hydrolysate.

40. The method of claim 37 wherein the enzymatic composition comprises at least one enzyme of Hypocrea jecorina.

41. The method of claim 37 wherein the plant biomass and the enzymatic composition are incubated at a temperature of from 27° C. to 37° C.

42. The method of claim 37 wherein the plant biomass and the enzymatic composition are incubated from 12 hours to 120 hours.

43. The method of claim 37 wherein the enzymatic composition comprises at least one enzyme of H. schweinitzii.

44. A method of preparing a plant biomass substrate for fermentation, the method comprising:
providing a plant biomass substrate;
heating the plant biomass substrate to a predetermined temperature of at least about 180° C. to no greater than about 300° C.;
pressurizing the plant biomass substrate to a predetermined pressure of at least about 200 psia to no greater than about 1000 psia;
maintaining the predetermined temperature and the predetermined pressure for a period of at least one minute and no greater than about 120 minutes;
cooling and depressurizing the plant biomass substrate;
providing an enzymatic composition comprising at least one enzyme capable of converting at least one of hemicellulose and cellulose to monomeric sugar; and
incubating the cooled plant biomass substrate with the enzymatic composition under conditions effective for the enzyme composition to convert at least one of hemicellulose and cellulose to monomeric sugars.

45. The method of claim 44 wherein the enzymatic composition comprises at least one enzyme of Hypocrea jecorina.

46. The method of claim 44 wherein the plant biomass substrate and the enzymatic composition are incubated at a temperature of from 27° C. to 40° C.

47. The method of claim 44 wherein the plant biomass substrate and the enzymatic composition are incubated from 12 hours to 120 hours.

48. The method of claim 44 wherein the enzymatic composition comprises at least one enzyme of H. schweinitzii.
